(19) United States
(12) Patent Application Publication
(10) Pub. No.: US 2009/0048666 A1
(43) Pub. Date: Feb. 19, 2009

(54) Medical devices having porous carbon adhesion layers

(75) Inventor: Tim O'Connor, Galway (IE)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc.

(21) Appl. No.: 11/893,417

(22) Filed: Aug. 14, 2007

(51) Int. Cl. A61F 2/06

(52) U.S. Cl. 623/1.39

(57) ABSTRACT

According to an aspect of the present invention, medical devices are provided which comprise a substrate, a porous carbon layer disposed on at least a portion of the substrate surface, and a polymeric layer disposed on at least a portion of the porous carbon layer.

### FIELD OF THE INVENTION

The present invention relates to medical devices and more particularly to medical devices having porous carbon surfaces.

### BACKGROUND OF THE INVENTION

Coronary stents such as those commercially available from Boston Scientific Corp. (TAXUS and PROMUS), Johnson & Johnson (CYPHER), and others are frequently prescribed use for maintaining blood vessel patency. These products are based on metallic balloon expandable stents with biostable polymer coatings, which release antirestenotic therapeutic agents at a controlled rate and total dose for preventing restenosis of the blood vessel. For example, the TAXUS stent coating is formed from biostable poly(styrene-b-isobutylene-b-styrene) triblock copolymer (SIBS) and employs paclitaxel as an antirestenotic agent.

One such device is schematically illustrated in FIGS. 1A and 1B. FIG. 1A is a schematic perspective view of a stent 100 which contains a number of interconnected struts 100s. FIG. 1B is a cross-section taken along line b-b of strut 100s of stent 100 of FIG. 1A and illustrates a stainless steel strut substrate 110 and a therapeutic-agent-containing polymeric coating 120, which encapsulates the entire stent strut substrate 110, covering the luminal 110l (blood contacting), abluminal 110a (vessel contacting), and side 110s surfaces thereof. Such a coating 120 need not have good adhesion to the stent substrate 110, because it is well-secured to the stent substrate 110 by encapsulation (assuming that the polymer has sufficient inherent strength).

### SUMMARY OF THE INVENTION

According to an aspect of the present invention, medical devices are provided which comprise a substrate, a porous carbon layer on at least a portion of the substrate surface, and a polymeric layer on at least a portion of the porous carbon layer.

The above and other aspects as well as various embodiments and advantages of the present invention will become immediately apparent to those of ordinary skill in the art upon review of the Detailed Description and Claims to follow.

### BRIEF DESCRIPTION OF THE DRAWINGS

FIG. 1A is a schematic perspective view of a stent in accordance with the prior art.

FIG. 1B is a schematic cross-sectional view taken along line b-b of FIG. 1A.

FIGS. 2A to 2C are schematic cross sectional views of stent struts, in accordance with various embodiments of the present invention.

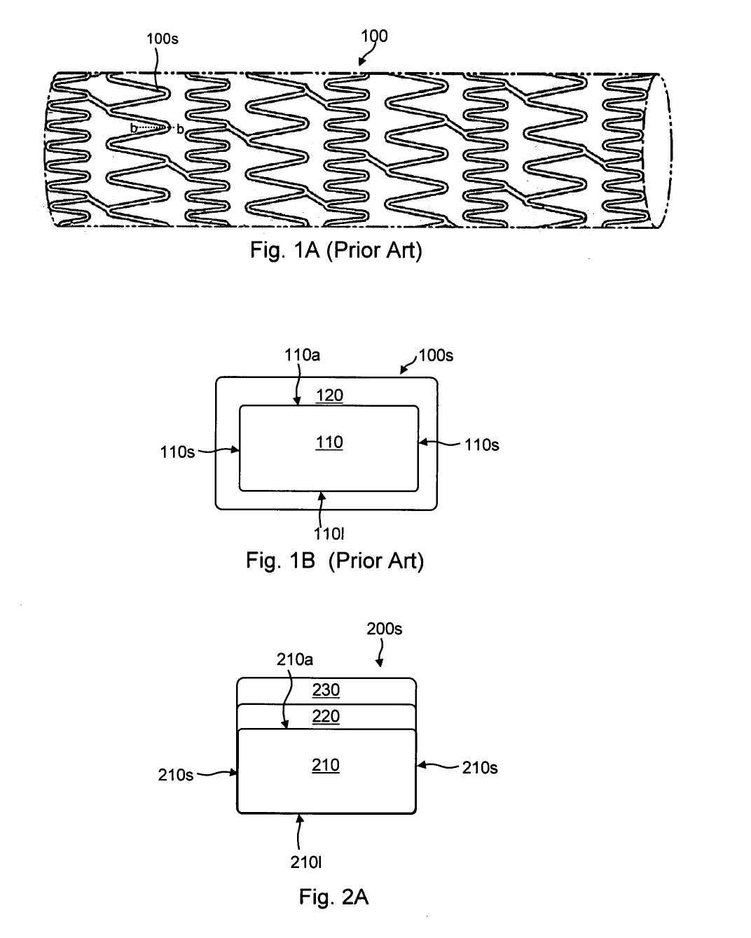

### DETAILED DESCRIPTION OF THE INVENTION

According to an aspect of the present invention, medical devices are provided which comprise a substrate, a porous carbon layer disposed on at least a portion of the substrate surface, and a polymeric layer disposed on at least a portion of the porous carbon layer.

As used herein a “layer” of a given material is a region of that material whose thickness is small compared to both its length and width. As used herein a layer need not be planar, for example, taking on the contours of an underlying substrate. A layer can be discontinuous (e.g., patterned). Terms such as “film,” “layer” and “coating” may be used interchangeably herein.

In certain embodiments, the polymeric layer further comprises a therapeutic agent. The porous carbon layer may also further comprise a therapeutic agent in certain embodiments, which may be the same as or different from the therapeutic agent of the polymeric layer. For example, a therapeutic agent may be disposed within the pores of the porous carbon layer, either with or without an added excipient (e.g., a polymer or mixture of polymers such as those described herein). “Therapeutic agents”, “pharmaceuticals,” “pharmaceutically active agents”, “drugs” and other related terms may be used interchangeably herein.

Some specific embodiments of the invention will now be described in conjunction with vascular stents, although it should be kept in mind that the invention is not so-limited and is applicable to a broad range of medical devices where good adhesion between a polymeric layer and an underlying substrate is desired.

In these embodiments pertaining to vascular stents, the porous carbon layer and the polymeric layer may be disposed, for example, over the abluminal surface of the stent substrate, but not the luminal and side surfaces, or the porous carbon layer and the polymeric layer may both be disposed over the luminal, abluminal and side surfaces of the stent substrate, or the porous carbon layer may be disposed over the luminal, abluminal and side surfaces of the stent substrate, while the polymeric layer is disposed over the abluminal surface of the stent substrate, but not the luminal and side surfaces, among other possibilities. As noted above, in some embodiments, the polymeric layer may further contain a therapeutic agent (e.g., an agent that inhibits the proliferation of smooth muscle cells) and/or the porous carbon layer may further contain a therapeutic agent (e.g., an agent that inhibits the proliferation of smooth muscle cells or an agent that reduces inflammatory responses).

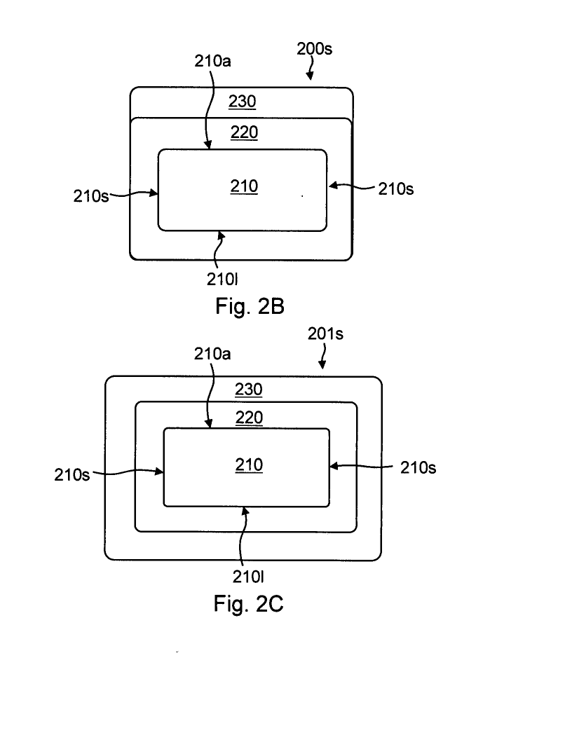

As a specific example, and with reference to the schematic cross sectional view of the stent strut 200s of FIG. 2A, a porous carbon layer 220 and a drug-eluting polymeric layer 230 may be provided over the abluminal surface 210a of the stent strut substrate 210, but not the luminal 210l and side 210s surfaces. In this embodiment, and without wishing to be bound to a particular theory of operation, it is believed that the porous carbon layer 220 acts primarily to enhance the adhesion of the polymeric layer 230 to the underlying stent strut substrate 210 (assuming that it does not contain a releasable therapeutic agent, etc.). Examples of materials for drug-eluting polymeric layer 230 include (a) an antirestenotic agent (e.g., paclitaxel, rapamycin, etc.) and (b) a polymer selected from poly(n-butyl methacrylate) homopolymers, poly(ethylene-co-vinyl acetate) copolymers, phosphoryl choline acrylate copolymers, poly(isobutylene-co-styrene) copolymers, poly(methyl methacrylate-co-n-butyl acrylate) copolymers, polylactide homopolymers, polyglycolide homopolymers, poly(lactide-co-glycolide) copolymers and poly(vinylidene fluoride-co-hexafluoropropylene) copolymers, among others.

As another example, with reference to FIG. 2B, the porous carbon layer 220 may be provided over the luminal 210l, abluminal 210a and side 210s surfaces of the stent strut substrate 210, whereas the drug-eluting polymeric layer 230 may again be provided over the abluminal surface 210a of the stent strut substrate 210, but not the luminal 210l and side 210s surfaces. Here, the porous carbon layer 220 acts to enhance the adhesion of the polymeric layer 230 to the abluminal surface 210a of the underlying stent strut substrate 210, and also provides a porous biocompatible surface over the luminal 210l and side 210s surfaces, which may optionally contain (and release) a further therapeutic agent. In the embodiment shown, the polymeric layer 220 does not extend over the side surfaces 210s of the substrate 210. In other embodiments, the polymeric layer is disposed over a small fraction (e.g., no more than 25%, more preferably no more than 10%, even more preferably no more than 5%) of the side surfaces. Such a small fraction may arise, for example, as a result of difficulties in manufacturing a polymeric layer that is not disposed over any portion of the side surfaces whatsoever. In still other embodiments, the polymeric layer is disposed of the abluminal and side surfaces of the stent substrate, but not the luminal surface.

Drug-eluting abluminal polymeric layers like those shown in FIGS. 2A and 2B have several advantages over currently marketed conformal polymeric coatings. First, the drug is located on the surface of the stent that is in contact with the blood vessel wall (i.e., the abluminal surface), which is where the therapeutic agent (e.g., an antiproliferative agent) is most needed. Moreover, because of the increased adhesion afforded by the porous carbon layer (discussed further below), a much reduced amount polymer is required to form a robust coating. Thus, the amount of implanted polymer is significantly reduced, minimizing the potential for inflammatory reaction to the polymer.

Turning now to FIG. 2C, a porous carbon layer 220 and a drug-eluting polymeric layer 230 may both be provided over the luminal 210l, abluminal 210a and side 210s surfaces of the stent strut substrate 210. Such an embodiment may be useful, for example, in the event that the polymeric layer 230 does not raise biocompatibility issues (e.g., inflammation, etc.) and/or in the event that encapsulation is not in and of itself sufficient to ensure that the polymeric layer 230 is well-secured to the stent substrate 210 (e.g., because the polymer lacks sufficient mechanical strength).

As noted above, the present invention is applicable to medical devices other than stents. Examples of medical devices benefiting from the present invention vary widely and include implantable or insertable medical devices, for example, stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts), vascular access ports, dialysis ports, catheters (e.g., urological catheters or vascular catheters such as balloon catheters and various central venous catheters), guide wires, balloons, filters (e.g., vena cava filters and mesh filters for distil protection devices), embolization devices including cerebral aneurysm filler coils (including Guglilmi detachable coils and metal coils), septal defect closure devices, myocardial plugs, patches, pacemakers, lead coatings including coatings for pacemaker leads, defibrillation leads, and coils, ventricular assist devices including left ventricular assist hearts and pumps, total artificial hearts, shunts, valves including heart valves and vascular valves, anastomosis clips and rings, cochlear implants, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, artificial ligaments, orthopedic prosthesis such as bone grafts, bone plates, fins and fusion devices, joint prostheses, orthopedic fixation devices such as interference screws in the ankle, knee, and hand areas, tacks for ligament attachment and meniscal repair, rods and pins for fracture fixation, screws and plates for craniomaxillofacial repair, dental implants, or other devices that are implanted or inserted into the body,

The medical devices of the present invention include, for example, implantable and insertable medical devices that are used for systemic diagnosis and treatment, as well as those that are used for the localized diagnosis and treatment of any tissue or organ. As used herein, “treatment” refers to the prevention of a disease or condition, the reduction or elimination of symptoms associated with a disease or condition, or the substantial or complete elimination of a disease or condition.

Substrate materials for the medical devices of the present invention may vary widely in composition and are not limited to any particular material, for example, being selected from biostable materials and biodisintegrable materials (i.e., materials that, upon placement in the body, are dissolved, degraded, resorbed, and/or otherwise removed from the placement site), organic and inorganic materials, and combinations of the foregoing. For example, substrate materials may be selected from (a) organic materials (i.e., materials containing organic species), for example, polymeric materials (i.e., materials containing polymers) such as suitable members of those set forth below for use in polymeric layers, (b) inorganic materials (i.e., materials containing inorganic species) including metallic inorganic materials (i.e., materials containing metals) and non-metallic inorganic materials (i.e., materials containing non-metallic inorganic materials), and (c) hybrid materials (e.g., hybrid organic/inorganic materials, for instance, polymer/metallic-inorganic hybrids and polymer/non-metallic-inorganic hybrids).

Inorganic materials (i.e., materials containing inorganic species, typically 50 wt % or more, for example, from 50 wt % to 75 wt % to 90 wt % to 95 wt % to 97.5 wt % to 99 wt % or more) may be selected, for example, from suitable metallic materials (i.e., materials containing metals, typically 50 wt % or more, for example, from 50 wt % to 75 wt % to 90 wt % to 95 wt % to 97.5 wt % to 99 wt % or more), which may be selected from the following: substantially pure metals including biostable metals such as gold, platinum, palladium, iridium, osmium, rhodium, titanium, tantalum, tungsten, and ruthenium, biodisintegrable metals such as magnesium and iron, biostable metal alloys such as alloys comprising iron and chromium (e.g., stainless steels, including platinum-enriched radiopaque stainless steel), niobium alloys, titanium alloys including alloys comprising nickel and titanium (e.g., nitinol), alloys comprising cobalt and chromium, including alloys that comprise cobalt, chromium and iron (e.g., elgiloy alloys), alloys comprising nickel, cobalt and chromium (e.g., MP 35N) and alloys comprising cobalt, chromium, tungsten and nickel (e.g., L605), alloys comprising nickel and chromium (e.g., inconel alloys), biodisintegrable metal alloys such as magnesium alloys, zinc alloys and iron alloys (including their combinations with each other, Ce, Ca, Zn, Zr and Li), metal and semi-metal oxides, including oxides silicon, aluminum, zirconium, titanium, iron, hafnium, tantalum, molybdenum, tungsten, rhenium, and iridium, and combinations of the foregoing, among many others.

Inorganic materials may further be selected, for example, from suitable non-metallic inorganic materials (i.e., materials containing non-metallic inorganic materials, typically 50 wt % or more, for example, from 50 wt % to 75 wt % to 90 wt % to 95 wt % to 97.5 wt % to 99 wt % or more) various metal- and non-metal-oxides (e.g., oxides of one or more of silicon, aluminum, titanium, zirconium, hafnium, tantalum, molybdenum, tungsten, rhenium, iron, niobium, and iridium), various metal- and non-metal-nitrides, various metal- and non-metal-carbides, various metal- and non-metal-borides, various metal- and non-metal-phosphates (e.g., calcium phosphate ceramics such as hydroxyapatite), various metal- and non-metal-sulfides, silicon and silicon-based ceramics such as those containing silicon nitrides, silicon carbides and silicon oxides (sometimes referred to as glass ceramics), among many others.

As noted above, in medical devices of the present invention, the substrate is at least partially covered with a porous carbon layer.

As used herein a “carbon layer” is one that contains at least 50 wt % carbon.

In certain embodiments, the carbon layer is a pyrolytic carbon layer. A “pyrolytic” material is one that is formed by pyrolysis (i.e., the decomposition of an organic material by heat, in a non-oxidizing environment). Because the resulting product is largely carbon, this process is also sometimes referred herein as a “carbonization” process and may be said to produce a “carbonized” product. Pyrolytic carbon has been used in medical prosthesis, particularly heart valves. See, e.g., F. J. Schoen et al., “Durability of pyrolytic carbon-containing heart valve prostheses,” J Biomed. Mater. Res. September 1982; 16(5):559-70.

In certain embodiments, the carbon layer is a carbon-carbon composite layer. As used herein a “carbon-carbon composite” layer is layer containing at least two forms of carbon, for example (among many other examples), a layer containing glassy/amorphous carbon and pyrolytic carbon. See Pub. No. US 2006/0177379 to Asgari.

The thickness of the carbon layers of the invention may vary widely, typically ranging from 100 nm to 250 nm to 500 nm to 1 μm to 2.5 μm to 5 μm to 10 μm to 20 μm or more in thickness. Where a pyrolyzed carbon layer is employed, the thickness of the layer will generally be a function of the thickness of the pre-pyrolysis precursor layer. In other words, the thickness of the pre-pyrolysis precursor layer will generally be proportional to the thickness of the pyrolyzed carbon layer.

The pore sizes of the porous carbon layers of the invention may vary widely. In many embodiments, the porous carbon layers are macroporous. As used herein, a “macroporous” region is one that contains pores that have pore widths that are greater than 50 nm, for example, ranging from 50 to 100 nm to 250 nm to 500 nm to 1 μm to 2.5 μm to 5 μm to 10 μm to 20 μm or more in width. Smaller pore sizes may limit the degree of penetration of the polymeric material into the pores at the time that the porous carbon layer is formed on the porous carbon layer (e.g., due to factors such as surface tension, coating viscosity, etc.). In some embodiments of the present invention, porous carbon layers are employed to enhance adhesion of the polymeric layer, for example, with the polymeric layer at least partially filling the pores of the porous carbon coating in a “lock and key” or “peg and hole” type arrangement.

By “polymeric layer” is meant a layer that comprises polymers, from 50 wt % or less to 75 wt % to 90 wt % to 95 wt % to 97.5 wt % to 99 wt % or more.

The thickness of the overlying polymeric layer can vary widely, typically ranging from 50 nm or less to 100 nm to 250 nm to 500 nm to 1 μm to 2.5 μm to 5 82 m to 10 μm to 20 μm or more in thickness. With respect to embodiments of the invention where polymeric layers are used to coat stent struts, the polymeric layer can be quite thin as encapsulation is not the basis for securing the polymeric layer to the underlying substrate material.

The polymer(s) within the polymeric layer may be biostable or biodisintegrable and may be selected, for example, from one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, and polysaccharides, including fibrin, fibrinogen, collagen, elastin, chitosan, gelatin, starch, and glycosaminoglycans such as hyaluronic acid; as well as blends and further copolymers of the above.

As indicated above, in some embodiments, the polymeric layer is a therapeutic-agent-eluting polymeric layer. As used herein, a “therapeutic-agent-eluting polymeric layer” is a layer that comprises a therapeutic agent and a polymer and from which at least a portion of the therapeutic agent is eluted upon implantation or insertion into a subject. Subjects are vertebrate subjects, more typically mammalian subjects, and include human subjects, pets and livestock. The therapeutic-agent-eluting polymeric layer will typically comprise, for example, from 1 wt % or less to 2 wt % to 5 wt % to 10 wt % to 25 wt % to 50 wt % or more of a single therapeutic agent or of a mixture of therapeutic agents within the layer. Therapeutic agents may be selected, for example, from those listed below, among others. The therapeutic-agent-eluting polymeric layer will also typically comprise, for example, from 50 wt % or less to 75 wt % to 90 wt % to 95 wt % to 97.5 wt % to 99 wt % or more of a single polymer or a mixture differing polymers within the layer.

As also indicated above, the porous carbon layer may also be associated with a therapeutic agent, which may be, for example, disposed within the pores of the porous carbon layer, disposed in a layer on the surface of the porous carbon layer, and so forth.

A wide variety of therapeutic agents may be employed in conjunction with the present invention, including genetic therapeutic agents, non-genetic therapeutic agents and cells, which may be used for the treatment of a wide variety of diseases and conditions. Numerous therapeutic agents are described here.

Suitable therapeutic agents for use in connection with the present invention may be selected, for example, from one or more of the following: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.), (z) selective estrogen receptor modulators (SERMs) such as raloxifene, lasofoxifene, arzoxifene, miproxifene, ospemifene, PKS 3741, MF 101 and SR 16234, (aa) PPAR agonists such as rosiglitazone, pioglitazone, netoglitazone, fenofibrate, bexaotene, metaglidasen, rivoglitazone and tesaglitazar, (bb) prostaglandin E agonists such as alprostadil or ONO 8815Ly, (cc) thrombin receptor activating peptide (TRAP), (dd) vasopeptidase inhibitors including benazepril, fosinopril, lisinopril, quinapril, ramipril, imidapril, delapril, moexipril and spirapril, (ee) thymosin beta 4, (ff) phospholipids including phosphorylcholine, phosphatidylinositol and phosphatidylcholine, and (gg) VLA-4 antagonists and VCAM-1 antagonists.

Preferred therapeutic agents include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the forgoing, among others.

Numerous therapeutic agents, not necessarily exclusive of those listed above, have been identified as candidates for vascular treatment regimens, for example, as agents targeting restenosis (antirestenotic agents). Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and αβ-antagonists such as labetalol and carvedilol, (e) endothelin receptor antagonists such as bosentan, sitaxsentan sodium, atrasentan, endonentan, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, atorvastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD (orgotein), SOD mimics, verteporfin, rostaporfin, AGI 1067, and M 40419, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) matrix metalloprotease (MMP) pathway inhibitors such as marimastat, ilomastat, metastat, pentosan polysulfate, rebimastat, incyclinide, apratastat, PG 116800, RO 1130830 or ABT 518, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate , nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, coichicine, Epo D, paclitaxel and epothilone), caspase activators, proteasome inhibitors, angiogenesis inhibitors (e.g., endostatin, angiostatin and squalamine), olimus family drugs (e.g., sirolimus, everolimus, tacrolimus, zotarolimus, etc.), cerivastatin, flavopiridol and suramin, (aa) matrix deposition/organization pathway inhibitors such as halofuginone or other quinazolinone derivatives, pirfenidone and tranilast, (bb) endothelialization facilitators such as VEGF and RGD peptide, and (cc) blood rheology modulators such as pentoxifylline.

Numerous additional therapeutic agents useful for the practice of the present invention are also disclosed in U.S. Pat. No. 5,733,925 to Kunz.

Various processes for forming medical devices in accordance with the invention will now be described. The present invention, however, is not limited to any particular process.

Among other attributes, the substrate selected should be able to withstand the conditions imposed by the carbon coating process. Once a suitable substrate is selected, a porous carbon coating may be formed on the same, for example, in accordance with the procedures described in Pub Nos. US 2005/0079200 to Rathenow and US 2006/0159718 to Rathenow. In general, the procedures described therein involve first providing a coating of a suitable organic material or composite material, which is then pyrolyzed.

Pyrolyzable organic materials may be selected, for example, from the following, among others: homopolymers or copolymers of polyvinyls such as polyvinyl chloride or polyvinyl alcohol, poly(meth)acrylic acid, polyacrylocyanoacrylate, polyacrylonitril, polyamides, polyesters, polyurethanes, polystyrene, polytetrafluoroethylene, biopolymers such as collagen, albumin, gelatin, hyaluronic acid and starch, celluloses such as methylcellulose, hydroxypropyl cellulose, hydroxypropyl methylcellulose, carboxymethylcellulose phthalate, waxes, casein, dextrans, polysaccharides, fibrinogen, polylactides, poly(lactide-co-glycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids, polyethylene vinyl acetate, poly(ester urethanes), poly(ether urethanes), poly(ester ureas), polyethers such as polyethylene oxide, polypropylene oxide, pluronics, polytetramethylene glycol, polyvinylpyrrolidone, poly(vinyl acetate phthalate) as well as their copolymers and mixtures of the same.

Typical temperatures for the pyrolysis step range from approximately 200° C. to approximately 1200° C., more typically, from 250° C. to 700° C. By suitably selecting and/or controlling the pyrolysis temperature, the porosity, the strength and the rigidity of the carbon coating, among other properties, may be adjusted in a controlled manner. In some embodiments, the temperature for the pyrolysis step is chosen such that the precursor film (e.g., polymer film, etc.) is transformed essentially completely into carbon-containing solids using as low a temperature as possible. The pyrolysis atmosphere is preferably an inert gas atmosphere, for example, selected from nitrogen, noble gases (e.g., helium, argon, neon, etc.) and combinations of the same.

Porosity may be generated using various techniques. For example, foaming polymer films, for instance, phenolic foams, polyolefin foams, polystyrene foams, polyurethane foams, and so forth, may be employed, which can then be converted into porous carbon layers in a subsequent pyrolysis step.

Porosity can also be induced by admixing volatile components which are degraded during carbonization and leave pores behind in the carbon-containing layer. For example, pores may be produced during carbonization using polymeric fillers that are substantially decomposed under carbonization conditions. In this regard, certain polymeric fillers, particularly saturated aliphatic hydrocarbons, may be decomposed substantially completely under carbonization conditions, reportedly based on processes analogous to petroleum cracking, to yield volatile hydrocarbons such as methane, ethane and the like, which then escape from the porous carbon framework of the carbonized layer during pyrolysis. Polymeric fillers may be selected, for example, from polyolefins such as polyethylene, polypropylene, polybutylene, polyisobutylene, polypentene as well as their copolymers and mixtures thereof. The pores produced may be dimensioned by a suitable choice of molecular weight, chain length and/or degree of branching of the polymeric fillers. Fillers may also be used in the form of particles, for example, thin fibers which may form suitably dimensioned pore passages during carbonization. For example, the porosity may be adjusted by selecting the fiber diameter and the fiber length, whereby larger fiber diameters and/or lengths may produce wider and longer pores. Such techniques may be suitable for producing porous layers having pore sizes in the range of about 10 nm to 100 μm.

Moreover, pre- or post-pyrolysis treatment steps such as reduction or oxidation processes may be carried out to modify pore size. For example, a carbon layer may be partially oxidized by exposure to an oxidizing gas atmosphere at elevated temperatures. Suitable oxidizing gases for partial oxidation include, for example, air, oxygen, carbon monoxide, carbon dioxide, nitrous oxide, and other oxidizing gases. The partial oxidation can be carried out at elevated temperatures, for example at temperatures ranging from 50 to 800° C. Partial oxidation may also be conducted using liquid agents at ambient or elevated temperatures, for example, liquid oxidizing agents such as acids (e.g., concentrated nitric acid) may be employed. Moreover, processes such as CVD (chemical vapor deposition) or CVI (chemical vapor infiltration) may be employed, in which the pore structure is modified by treatment with suitable gases that split off carbon at high temperatures or gases which deposit carbon, thereby alone one to enlarge or reduce the pore size.

As further examples, porous composite layers can be formed in accordance with the procedures of Pub. Nos. US 2007/0003749 and US 2007/0003753 to Asgari, followed by pyrolysis of such structures.

Porous carbon coated substrates can also be obtained commercially, for example, from companies such as Blue Membranes GmbH. The pore size of carbon coatings produced by Blue Membranes GmbH can be fixed between a minimum of about 50 nm and a maximum of about 10 μm.

Once a porous carbon layer is formed, therapeutic-agent-eluting polymeric layers may be disposed over the porous-carbon-coated substrate using any suitable method known in the art. For example, where the polymeric layer contains one or more polymers having thermoplastic characteristics, the layer may be formed, for instance, by (a) providing a melt that contains polymer(s) and any other optional species desired such as therapeutic agent(s) and (b) applying the melt to the porous-carbon-coated substrate. After application, the melt may be cooled actively (e.g., by applying a chilled stream of air) or passively. As another example, a layer may be formed, for instance, by (a) providing a solution or dispersion that contains one or more solvent species, polymer(s), and any other optional species desired such as therapeutic agent(s) and (b) applying the solution or dispersion to the porous-carbon-coated substrate. After application, the solvent is removed actively (e.g., by applying heat and/or vacuum) or passively (e.g., by allowing evaporation to occur under ambient conditions). Similar procedures may be used to apply therapeutic agent(s) to the porous-carbon-coated substrate in the absence of polymer as well.

The viscosity of the melt may be lowered (e.g., to enhance penetration into the pores of the carbon coating) by increasing the application temperature, among other techniques. The viscosity of the solution or dispersion may be lowered by increasing the application temperature and/or decreasing the polymer concentration, among other methods. The surface tension of the solution or dispersion may be varied (e.g., to enhance penetration into the pores of the carbon coating) by changing the solvent composition or by changing the temperature. Increasing the surface energy on the carbon coating by plasma treatment may enhance the penetration of the polymer layer.

The melt, solution or dispersion may be applied, for example, by roll-coating a porous-carbon-coated substrate (e.g., where it is desired to apply the layer to the abluminal surface of a tubular device such as a stent), by application to the porous-carbon-coated substrate using a suitable application device such as a brush, roller, stamp or ink jet printer, or by dipping or spray coating the porous-carbon-coated substrate, among other methods. In certain techniques (e.g., dipping, spraying, etc.), a portion of the porous-carbon-coated substrate may be masked to prevent the polymeric layer from being applied thereon.

### EXAMPLE 1

As one specific example, a stainless steel stent may be coated with a coating of carbon-carbon composite material in accordance with Example 4 of Pub. No. US 2006/0177379 to Asgari. Briefly, 16 mm Liberté™ stainless steel stents are cleaned with an RF oxygen plasma in a March AP-1000 Plasma System using the following process parameters: P=200 mTorr, 300 watts, Gas 1 (Argon)=250 sccm, Gas2 (Oxygen)=200 sccm, t=180 s. A dispersion of a phenoxy resin, Beckopox EP 401 (from UCB Company), commercially available carbon black, Printex alpha (from Degussa), and a fullerene mixture of C60 and C70 (from FCC Company, sold as Nanom-Mix), in methylethylketone, is prepared. The solids content of the polymer is 0.5 wt %, the solids content of carbon black is 0.3 wt %, the solids content of the fullerene mix is 0.2 wt %, and the solvent accounts for 99 wt % of the dispersion. The precursor solution is sprayed onto the stent substrates as a precursor film and tempered by application of hot air at 350° C. in ambient air. Then the sample is treated thermally in a commercial tube reactor, under nitrogen atmosphere with a heat-up and cool-down ramp of 1.3 K/min, a holding temperature of 300° C., and a holding period of 30 minutes. The sample is treated in an ultrasonic bath in 10 ml of a 50% ethanol solution at 30° C. for 20 minutes, washed, and dried in a commercial convection oven at 90° C. This procedure is reported to produce a coating of a glass-like amorphous carbon/pyrolytic carbon having a surface area weight of 1.75 g/m² and an average pore size of about 1 μm.

The abluminal surface of the stent is then coated by roll-coating the stent in a viscous solution which contains, for example, SIBS, paclitaxel and toluene or a viscous solution of PVDF, everolimus, acetone and cyclohexanone among others.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device comprising a substrate, a porous carbon layer disposed on the substrate surface, and a polymeric layer disposed on the porous carbon layer.

2. The medical device of claim 1, wherein the substrate is a metallic substrate.

3. The medical device of claim 1, wherein the porous carbon layer is a pyrolytic carbon layer.

4. The medical device of claim 1, wherein the porous carbon layer is a porous carbon-carbon composite layer.

5. The medical device of claim 1, wherein said porous carbon layer is a macroporous layer.

6. The medical device of claim 1, wherein said porous carbon layer comprises pores having widths of 1 μm or more.

7. The medical device of claim 1, wherein the porous carbon layer further comprises a therapeutic agent.

8. The medical device of claim 1, wherein the therapeutic agent is selected from an antiproliferative agent, an antirestenotic agent, and an anti-inflammatory agent.

9. The medical device of claim 1, wherein the polymeric layer comprises a biostable polymer.

10. The medical device of claim 1, wherein the polymeric layer comprises a biodisintegrable polymer.

11. The medical device of claim 1, wherein the polymeric layer comprises a polymer selected from poly(n-butyl methacrylate) homopolymers, poly(ethylene-co-vinyl acetate) copolymers, phosphoryl choline acrylate copolymers, poly(isobutylene-co-styrene) copolymers, poly(methyl methacrylate-co-n-butyl acrylate) copolymers, polylactide homopolymers, polyglycolide homopolymers, poly(lactide-co-glycolide) copolymers and poly(vinylidene fluoride-co-hexafluoropropylene).

12. The medical device of claim 1, wherein the polymeric layer further comprises a therapeutic agent.

13. The medical device of claim 14, wherein the therapeutic agent is selected from an antiproliferative agent and an antirestenotic agent.

14. The medical device of claim 14, wherein the therapeutic agent is selected from paclitaxel, sirolimus, everolimus, tacrolimus and zotarolimus.

15. The medical device of claim 1, wherein the combined thickness of the porous carbon layer and the polymeric layer ranges from 1 μm to 10 μm.

16. The medical device of claim 1, wherein the substrate is a vascular stent that comprises luminal and abluminal surfaces.

17. The medical device of claim 16, wherein said polymeric layer further comprises an antirestenotic agent.

18. The medical device of claim 17, wherein the porous carbon layer covers the entire surface of the vascular stent and wherein the polymeric layer is disposed over the abluminal surface of the stent but not the luminal surface.

19. The medical device of claim 17, wherein the porous carbon layer and the polymeric layer are disposed over the abluminal surface of the stent but not the luminal surface.

20. The medical device of claim 17, wherein the porous carbon layer and the polymeric layer are each disposed over the entire surface of the vascular stent.
